(19) United States
(12) Patent Application Publication
(10) Pub. No.: US 2009/0171344 A1
(43) Pub. Date: Jul. 2, 2009

(54) APPARATUS AND METHODS FOR MONITORING PATIENT-APPARATUS CONTACT

(76) Inventor: GEORGE PONTIS, Redwood City, CA (US)

Correspondence Address: Cutera c/o MoFo, 425 Market Street, San Francisco, CA, 94105-2482, US

(21) Appl. No.: 11/964,427

(22) Filed: Dec. 26, 2007

(51) Int. Cl. A61B 18/04

(52) U.S. Cl. 606/35

(57) ABSTRACT

Systems, apparatus and methods for monitoring contact of electrosurgical apparatus with a patient's body via a contacting monitoring unit having a plurality of contacting segments, wherein each of the contacting segments is separate from an active electrode and/or return electrode. In an embodiment, the contacting monitoring unit may sense an electrical parameter value of the contacting segments to indicate contact, or lack of contact, of each contacting segment with the patient's body.

### FIELD OF THE INVENTION

The present invention generally relates to apparatus and methods for monitoring contact of an electrosurgical apparatus with a patient.

### BACKGROUND OF THE INVENTION

Various forms of electrosurgery are now widely used for a vast range of surgical procedures. There are two basic forms or electrosurgery, namely monopolar and bipolar, according to the configuration of the electrosurgical system which determines the path of electrical energy flow vis-à-vis the patient. In the bipolar configuration, both the active electrode and the return electrode are located adjacent to a target tissue of the patient, i.e., the electrodes are in close proximity to each other, and current flows between the electrodes locally at the surgical site. In monopolar electrosurgery, the active electrode is again located at the surgical site; however, the return electrode, which is typically much larger than the active electrode, is placed in contact with the patient at a location on the patient's body that is remote from the surgical site. In monopolar electrosurgery, the return electrode is typically accommodated on a device which may be referred to as a dispersive pad, and the return electrode may also be known as the, dispersive-, patient-, neutral-, or grounding electrode.

In general, monopolar electrosurgical procedures allow a large range of tissue effects. In monopolar electrosurgery, current from an electrosurgical generator typically flows through an active electrode and into target tissue. The current then passes through the patient's body to the return electrode where it is collected and returned to the generator.

A disadvantage of monopolar electrosurgery using prior art return electrodes is the risk of burns on the patient's body at the location of the return electrode. In the case of a solid return electrode, e.g., a metal plate, electric current density tends to be concentrated at the corners and/or edges of the return electrode. Concentration, or uneven distribution, of electric current density at the return electrode surface may cause excessive heating to the extent that a severe burn to the patient's tissue can result. Dislocation of the return electrode, e.g., resulting in only partial contact of the return electrode with the patient's body, can result in local increase(s) in electric current density, which again may result in a patient burn.

Some newer electrosurgical systems and applications use substantially higher current values, higher duty cycles, and/or longer delivery times for ablating, heating, or modifying target tissue, as compared with more traditional uses of electrosurgery. With these higher current densities and longer delivery times, the risk of a patient burn, from either a return electrode or an active electrode, may be greatly increased. The present IEC 60601-2-2:2006 standard states that “No acceptable neutral electrode should exceed a 6° C. temperature rise when subjected to the required current and duration test.” The Association for the Advancement of Medical Instrumentation (“AAMI”) has published similar standards.

A partial dislodgment of a dispersive pad from the patient's body, resulting in incomplete contact of the return electrode with the patient's body, increases the risk of a patient burn due to the resulting higher current density. Therefore, various mechanisms or circuits have been used to monitor contact between the return electrode and the patient. For example, U.S. Pat. No. 3,683,923 to Anderson discloses a circuit in which the input current to an active electrode is compared with the output current from a return electrode plate; lack of equality generates an error signal. In another embodiment of the '923 patent, the equality of output currents from two plates of a return electrode is sensed; lack of equality again causes an error signal.

U.S. Pat. No. 4,657,015 to Inrich discloses a control electrode affixed to the patient's body during a procedure for sensing a voltage on the body resulting from lack of contact between a return electrode and the patient.

U.S. Patent Application Publication No. 20070167942 (Rick) discloses a return pad current distribution system including a return electrode having a plurality of conductive elements. At least one sensor measures current levels returning to each conductive element of the return electrode, the current levels are input to a computer algorithm, and a variable impedance controller adjusts variable impedance levels to portions of the return electrode based on output from the computer algorithm.

In addition to problems associated with lack of contact of a return electrode with the patient, as outlined above, lack of contact between a patient's body and one or more active electrodes of an electrosurgical instrument may also be undesirable. For example, lack of contact of an active electrode with a treatment area may result in an uneven treatment of a target tissue or a mottled tissue effect, and/or may result in localized high current density with the concomitant risk of a patient burn.

As can be seen, there is a need for apparatus and methods for reliably monitoring contact between apparatus having a return electrode and the patient's body during electrosurgery.

There is a further need for apparatus and methods for monitoring contact between apparatus having an active electrode and the patient's body.

### SUMMARY OF THE INVENTION

According to one aspect of the invention, there is provided a system for monitoring contact of an apparatus with a patient's body. The system includes an electrode configured for contacting the patient's body, and a contact monitoring unit including a plurality of contacting segments. Each contacting segment is physically separate from the electrode, and the contact monitoring unit is configured for monitoring contact between each contacting segment and the patient's body.

According to a further aspect of the invention, a system for monitoring contact with a patient's body includes a contact monitoring unit including a plurality of contacting segments, and an electrode configured for contacting the patient's body. Each of the contacting segments is disposed adjacent to the electrode, and each contacting segment is configured for contacting the patient's body independently of the electrode. The contacting segments are capacitively coupled to the electrode, and the system is configured for sensing a change in voltage of the contacting segments responsive to loss of contact of the electrode with the patient's body.

According to another aspect of the invention, a dispersive return pad comprises a return electrode configured for contacting a patient's body, and a contacting element including a plurality of contacting segments. The contacting segments are disposed adjacent to the return electrode, and each of the contacting segments is configured for contacting the patient's body. Each of the contacting segments is physically separate from the return electrode, and the contacting element is configured for monitoring contact between the contacting segments and the patient's body.

According to still another aspect of the invention, an apparatus for treating a patient comprises an active electrode configured for applying electrical energy to the patient's body, and a plurality of contacting segments disposed adjacent to the active electrode. Each of the contacting segments is physically separate from the active electrode, and the apparatus is configured for monitoring contact between each contacting segment and the patient's body.

According to yet another aspect of the invention, a method for monitoring an electrosurgical procedure comprises contacting the patient's body with an electrode; contacting the patient's body with a plurality of contacting segments; and monitoring contact between each contacting segment and the patient's body, wherein each contacting segment is physically separate from the electrode.

These and other features, aspects, and advantages of the present invention may be further understood with reference to the drawings, description, and claims which follow.

### BRIEF DESCRIPTION OF THE DRAWINGS

FIG. 1 schematically represents a monopolar electrosurgical system, including a return electrode and an active electrode, according to an embodiment of the invention;

FIG. 2 is a block diagram schematically representing an electrosurgical system including a contact monitoring unit, according to another embodiment of the invention;

FIG. 3 is a block diagram schematically representing a contact monitoring unit, according to another embodiment of the invention;

FIG. 4 is a block diagram schematically representing an electrosurgical system having a contact sensing module and a power supply, according to an embodiment of the invention;

FIG. 5A schematically represents an electrosurgical system configured for monitoring contact between a patient and an electrode-bearing apparatus, according to an embodiment of the invention;

FIG. 5B schematically represents an electrosurgical system configured for monitoring contact between a patient and an electrode-bearing apparatus, according to another embodiment of the invention;

FIG. 6A schematically represents a dispersive return pad having a plurality of contacting segments, according to another embodiment of the invention;

FIG. 6B schematically represents a treatment face of an electrosurgical instrument having a plurality of contacting segments, according to another embodiment of the invention;

FIG. 7A schematically represents an electrosurgical apparatus having a return electrode shown in relation to a patient's body, according to another embodiment of the invention;

FIG. 7B schematically represents an electrosurgical apparatus having an active electrode shown in relation to a patient's body, according to another embodiment of the invention;

FIG. 8 is a flow chart schematically representing a series of steps involved in a method for monitoring contact between a dispersive return pad and a patient's body during an electrosurgical procedure, according to another embodiment of the invention; and

FIG. 9 is a flow chart schematically representing a series of steps involved in a method for monitoring contact between an electrosurgical instrument and a patient's body during an electrosurgical procedure, according to another embodiment of the invention.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

Broadly, the present invention provides apparatus and methods for monitoring contact between a patient's body and an electrode-bearing structure. The electrode-bearing structure may be, for example, a component of an electrosurgical apparatus, and contact with the patient's body may be monitored during a procedure. The electrosurgical apparatus may be, for example, one or both of: an electrosurgical instrument having at least one active electrode, or a dispersive return pad having at least one return electrode. The electrode-bearing structure of the apparatus may be, for example, a treatment face of an electrosurgical instrument or a support layer of a dispersive return pad. The methods and apparatus of the instant invention may find applications in reliably monitoring patient contact with such electrosurgical apparatus during a broad range of electrosurgical procedures. Such procedures may involve, for example, cutting and/or coagulation during general surgery, as well as various cosmetic procedures, and the like.

Prior art electrosurgical monitoring apparatus and methods have used various devices and/or circuitry for directly monitoring contact between a return electrode and the patient's body. In contrast to the prior art, the instant invention provides apparatus having separate contacting segments, physically separate from the electrode(s), disposed on an electrode-bearing surface or layer, and dedicated to monitoring contact of the contacting segments with the patient's body. The contacting segments may be disposed on an electrode support structure, such as a support layer of a dispersive pad or a treatment face of an electrosurgical instrument, for indirectly monitoring contact of the electrode support structure with the patient. Accordingly, the instant invention allows identification of a potentially unfavorable condition of an electrode support structure, vis-à-vis the patient's body, before an electrode supported by the electrode support structure has lost contact with the patient.

In further contrast to the prior art, contact monitoring of the present invention is applicable to both electrosurgical instruments (active electrodes) and dispersive pads (return electrodes). Furthermore, since the contacting segments are physically separate from the electrode, the instant invention is not constrained by electrode configuration.

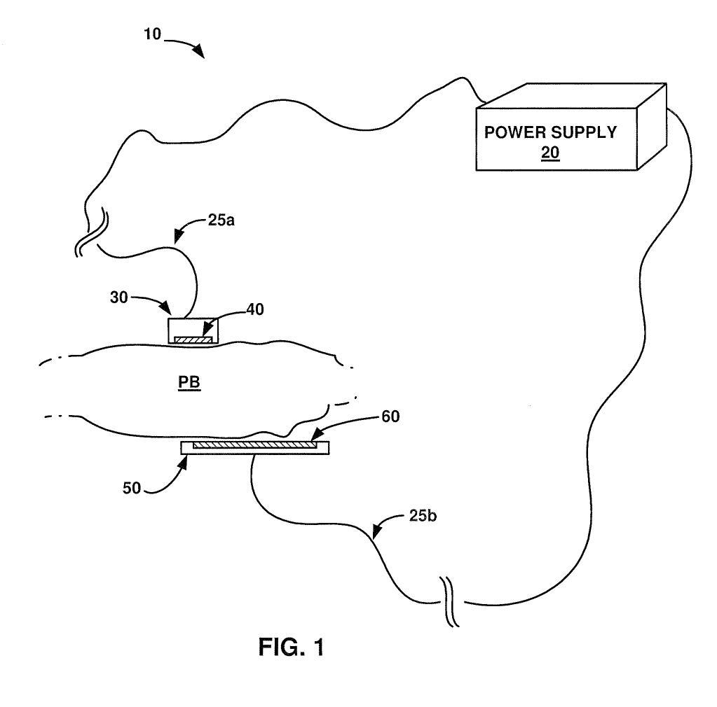

FIG. 1 schematically represents a monopolar electrosurgical system including a return electrode and an active electrode, according to an embodiment of the invention. System 10 may include an electrosurgical generator or power supply 20, an electrosurgical instrument 30, a dispersive return pad 50, a first cable 25a, and a second cable 25b. Electrosurgical instrument 30 may include an active electrode 40 configured for applying electrical energy from power supply 20 to the patient's body, PB. Dispersive return pad 50 may include a return electrode 60 configured for receiving the electrical energy from the patient's body. Active electrode 40 may be coupled to power supply 20 via first cable 25a for supplying the electrical energy to active electrode 40. Return electrode 60 may be coupled to power supply 20 via second cable 25b for returning the electrical energy to power supply 20.

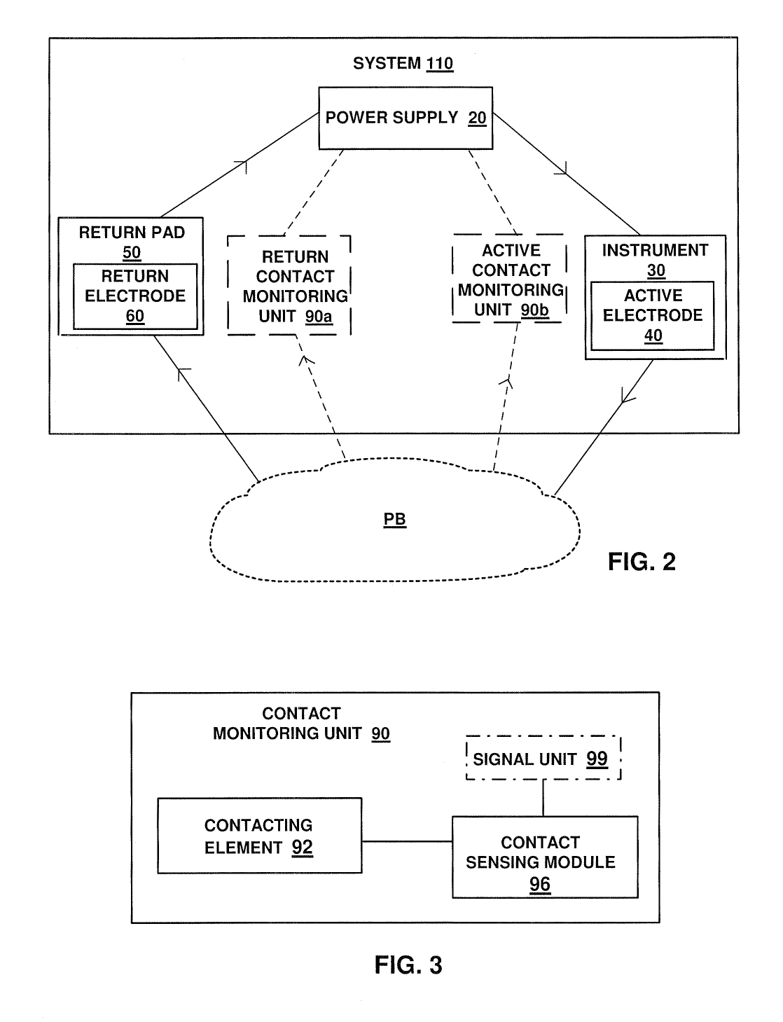

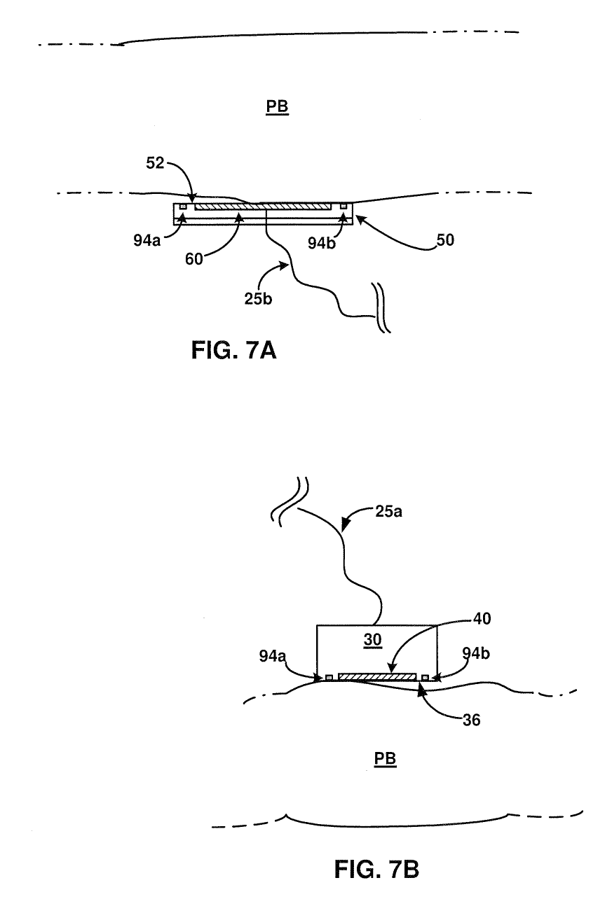

In an embodiment, electrosurgical instrument 30 may be configured for monitoring contact between at least a portion of electrosurgical instrument 30 and the patient's body. In another embodiment, dispersive return pad 50 may be configured for monitoring contact between at least a portion of dispersive return pad 50 and the patient's body. In another embodiment, both of electrosurgical instrument 30 and dispersive return pad 50 may be configured for monitoring contact of both electrosurgical instrument 30 and dispersive return pad 50 with the patient's body (see, e.g., FIGS. 2 and 7A-B).

FIG. 2 is a block diagram schematically representing an electrosurgical system 110 shown in relation to a patient's body, according to an embodiment of the invention. System 110 may include a power supply 20, a dispersive return pad 50, an electrosurgical instrument 30, a return contact monitoring unit 90a, and an active contact monitoring unit 90b. Dispersive return pad 50 and electrosurgical instrument 30 may include a return electrode 60 and an active electrode 40, respectively (see, e.g., FIGS. 1, and 7A-B). Electrical energy from power supply 20 may be applied to a patient's body via active electrode 40. Return electrode 60 may receive the electrical energy from the patient's body for return of the electrical energy to power supply 20.

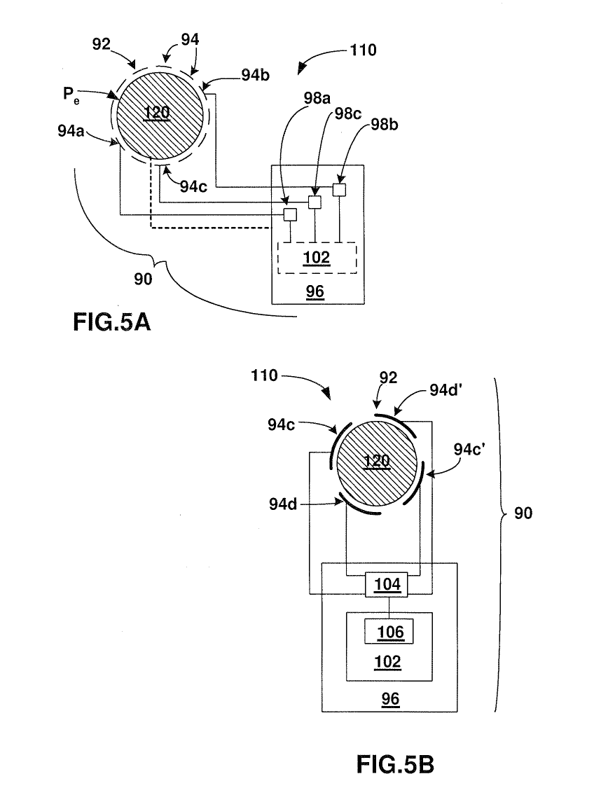

In an embodiment, return contact monitoring unit 90a may be configured for monitoring contact between at least a portion of dispersive return pad 50 and the patient's body (see, e.g., FIGS. 5A-B, 6A, and 7A). Similarly, active contact monitoring unit 90b may be configured for monitoring contact between at least a portion of electrosurgical instrument 30 and the patient's body (see, e.g., FIGS. 5A-B, 6B, and 7B).

Active contact monitoring unit 90b may be included in system 110 in addition to, or instead of, return contact monitoring unit 90a. Thus, in various embodiments, one or both of dispersive return pad 50 and electrosurgical instrument 30 may be monitored by return contact monitoring unit 90a or active contact monitoring unit 90b, respectively.

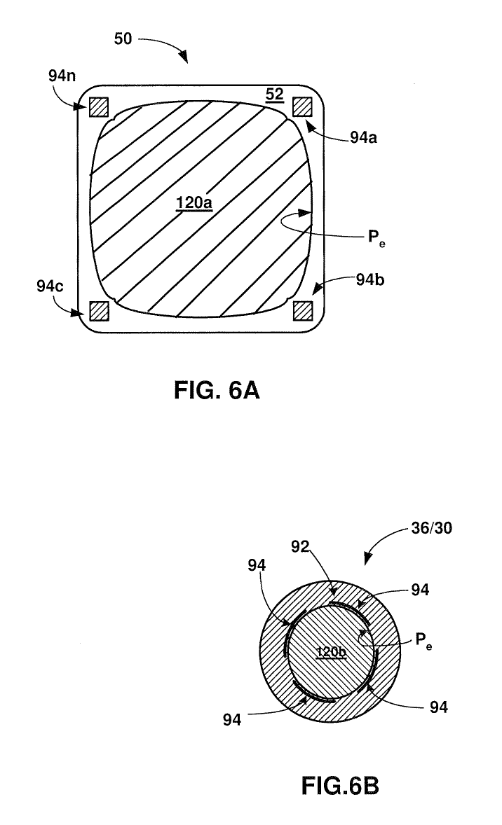

Each of contact monitoring units 90a and 90b may be in communication with, or electrically coupled to, power supply 20 for controlling, e.g., shutting off, power supply 20 if there is a lack of contact or only partial contact between a patient-contacting portion of dispersive return pad 50 or electrosurgical instrument 30 and the patient's body. A patient-contacting portion of dispersive return pad 50 may comprise, as an example, a support layer 52 bearing a plurality of contacting segments 94a-n (see, e.g., FIG. 6A). A patient-contacting portion of electrosurgical instrument 30 may comprise, as an example, a treatment face 36, wherein treatment face 36 may have a plurality of contacting segments 94 disposed thereon (see, e.g., FIG. 6B).

FIG. 3 is a block diagram schematically representing a contact monitoring unit, according to another embodiment of the invention. Contact monitoring unit 90 of FIG. 3 may include a contacting element 92 and a contact sensing module 96. Contacting element 92 may include a plurality of contacting segments 94 (see, for example, FIGS. 5A-B). Contacting segments 94 may be configured for contacting the patient's body. Contact sensing module 96 may include one or more contact sensors (see, for example, FIG. 5A). In an embodiment, contact sensing module 96 may be configured for sensing a value of at least one electrical parameter for each of contacting segments 94.

Typically, an amount of any electrical energy received from or delivered to the patient's body by contacting element 92 will be much less than that received or delivered by return electrode 60 and active electrode 40. For practical purposes, reception or delivery of any electrical energy to or from the patient's body by contacting element 92 may be considered a by-product of their contact monitoring function.

In some embodiments, contact monitoring unit 90 may further include a signal unit 99 for signaling, e.g., to medical personnel, a lack of contact between one or more contacting segments 94 and a patient's body. Signal unit 99 may be in communication with contact sensing module 96 for actuation of signal unit 99 in response to a lack of contact between at least one contacting segment 94 and the patient's body. Upon actuation, signal unit 99 may provide a signal to medical personnel, e.g., an audible signal and/or a visual signal, and the like.

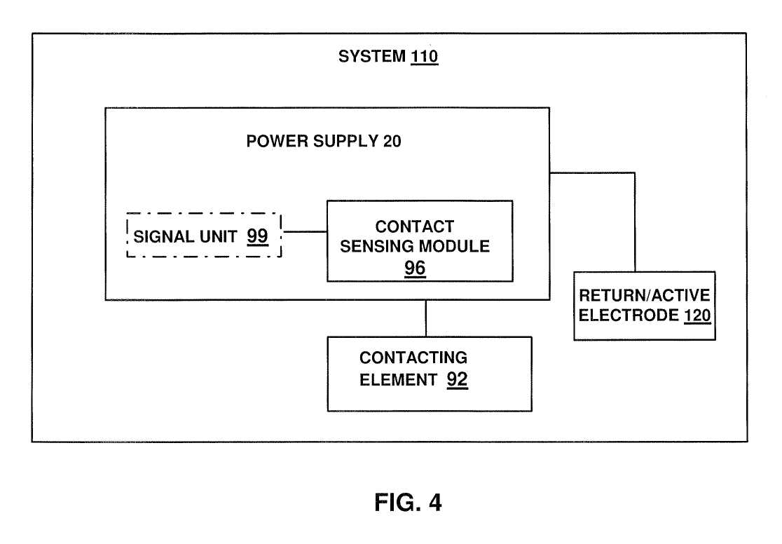

FIG. 4 is a block diagram schematically representing an electrosurgical system 110, according to another embodiment of the invention. System 110 of FIG. 4 may include a power supply 20, a contacting element 92 in communication with power supply 20, and at least one electrode 120, wherein the electrode 120 may be a return electrode (see, e.g., FIGS. 6A, 7A) and/or an active electrode (see, e.g., FIGS. 6B, 7B). As shown, a contact sensing module 96 and signal unit 99 may be disposed or housed within power supply 20. In alternative embodiments, signal unit 99 may be integral with an electrosurgical instrument 30, and the like. A location, or component, for housing signal unit 99 may depend on whether signal unit 99 provides a visual or audible signal. Of course, some embodiments may have more than one signal unit 99 configured to provide either an audible signal or a visual signal, or both an audible signal and a visual signal.

FIG. 5A schematically represents an electrosurgical system, according to an embodiment of the invention. Electrosurgical system 110 of FIG. 5A may include at least one electrode 120 and a contact monitoring unit 90. Contact monitoring unit 90 may include a contacting element 92 and a contact sensing module 96. Contacting element 92 may be configured for monitoring contact between contacting segments 94 and a patient's body during an electrosurgical procedure. In an embodiment, electrode 120 may comprise or represent a return electrode (see, e.g., FIGS. 6A, 7A). In other embodiments, electrode 120 may comprise or represent an active electrode (see, e.g., FIGS. 6B, 7B).

Contacting element 92 may comprise a plurality of contacting segments 94. Electrosurgical system 110 may be configured for directly monitoring contact of each of contacting segments 94 with the patient's body. Lack of contact between at least one contacting segment 94 and the patient's body may indicate a situation which could lead to either complete or partial loss of contact between electrode 120 and the patient's body, whereby such an indication may allow intervention by medical personnel to prevent any loss of contact between electrode 120 and the patient's body.

Each of contacting segments 94 may be physically isolated or separate from electrode 120. The plurality of contacting segments 94 may also be physically isolated or separate from each other. Each of contacting segments 94 may be configured for contacting a patient's body, and each of contacting segments 94 may contact the patient's body independently of each other and independently of electrode 120.

Typically, an amount of any electrical energy received from or delivered to the patient's body by contacting segments 94 will be much less than that received or delivered by electrode 120. For example, a first electrical power level of contacting segments 94 may be less than a second electrical power level of electrode 120. Typically, electrical energy received from or delivered to the patient's body by contacting segments 94 will be less than 50 percent (<50%) that received or delivered by electrode 120, usually less than 10 percent (<10%) that received or delivered by electrode 120, and often less than one percent (<1%) that received or delivered by electrode 120. In this regard, it should be clearly understood that contacting segments 94 are not intended to function as either active electrodes or return electrodes.

By monitoring patient contact with contacting segments 94, each contacting segment 94 may be used to monitor patient contact with a portion of an electrode-bearing structure disposed adjacent to the particular contacting segment 94. In the case of a dispersive return pad 50, such an electrode-bearing structure may comprise a support layer 52 (see, e.g., FIG. 6A), while in the case of an electrosurgical instrument 30, such an electrode-bearing structure may comprise a treatment face 36 of instrument 30 (see, e.g., FIG. 6B).

Contacting segments 94 may typically be disposed adjacent to electrode 120. In this context, “adjacent to” may be used: i) to describe a configuration wherein one or more of contacting segments 94 may be disposed external to a zone bounded by the perimeter, Pₑ, of electrode 120, e.g., radially outward from the electrode perimeter, substantially as shown in FIGS. 5A-B; as well as, i.e., additionally or alternatively, ii) to describe a configuration wherein one or more of contacting segments 94 may be disposed within a zone bounded by the perimeter of electrode 120 (the latter configuration is not shown). In an embodiment, contacting segments 94 may at least partially encircle or surround electrode 120.

During use of electrode 120, if one or more of contacting segments 94 loses contact with the patient's body, a difference or deviation in electrical energy distribution between contacting segments 94 may be apparent. Such difference(s) in electrical energy distribution may be detected, e.g., via contact sensing module 96, for the purpose of monitoring contact between each of contacting segments 94 and the patient's body.

Since contacting segments 94 may contact the patient's body independently of electrode 120, e.g., contacting segments 94 may be disposed external to a zone bounded by the perimeter of electrode 120, lack of contact between one or more contacting segments 94 and the patient's body may be detected, and corrective action may be taken, before any lack of contact between electrode 120 and the patient's body occurs. Stated differently: since contacting segments 94 may contact the patient's body independently of electrode 120, detection of lack of contact between one or more contacting segments 94 and the patient's body allows corrective action to prevent any lack of contact between electrode 120 and the patient's body.

Contact sensing module 96 may include at least one contact sensor, and typically a plurality of contact sensors, of which three (3) contact sensors 98a-c are shown in FIG. 5A. Other numbers and configurations of contact sensors are also contemplated under the invention. Contacting segments 94 may be in electrical communication with, e.g., electrically coupled to, contact sensing module 96. In a non-limiting example, as shown in FIG. 5A, each of contacting segments 94a-c may be in electrical communication with a corresponding one of contact sensors 98a-c. Each of contact sensors 98a-c may be configured for sensing at least one electrical parameter, such as current, voltage, impedance, or power, of a corresponding one of contacting segments 94a-c. The nature of the electrical parameter(s) to be sensed according to various embodiments of the invention may be selected, for example, depending on the configuration of system 110. Herein, the term “voltage” may be used to refer to electric potential, as is commonly accepted in the art.

With reference once again to FIG. 5A, system 110 may still further include a comparator 102 in communication with each of contact sensors 98a-c. As an example, comparator 102 may comprise a microcontroller or a microprocessor, and the like. A significant change in the sensed value of an electrical parameter for one or more of contacting segments 94 may indicate that any contacting segment(s) 94 having experienced such a change may lack contact with the patient's body.

According to one embodiment of the present invention to be described with reference to FIG. 5A, comparator 102 may be configured or programmed for comparing a sensed value of an electrical parameter of each contacting segment 94 against a pre-set or reference value of that electrical parameter. Such a pre-set or reference value may be a threshold level, or a mathematical function thereof, above or below a normal range of the electrical parameter typically observed during contact of contacting segment(s) 94 with the patient's body. In an embodiment, a sensed electrical parameter value above or below the threshold level, e.g., below a minimum threshold level for the electrical parameter, for any one of contacting segments 94, may indicate lack of contact between that contacting segment 94 and the patient's body. The sensed electrical parameter(s) may be, for example, current, voltage, power, or impedance.

As a non-limiting example, lack of contact between a contacting segment 94 and the patient's body may typically cause a change (e.g., a discernible decrease or increase) in current and voltage. As a non-limiting example, lack of contact between a contacting segment 94 and the patient's body may be detected, via a contact sensor 98a, 98b, or 98c, as a decrease in current or voltage values below the threshold level. In an embodiment, the reference value or threshold level may be selected based on a number of factors such as the size of the electrosurgical apparatus bearing contacting segments 94, the nature of the procedure, and the like.

According to another embodiment to be described with reference to FIG. 5A, comparator 102 may be configured for comparing a sensed value of an electrical parameter for one or more contacting segments 94 with a sensed value of the electrical parameter for one or more other contacting segments 94. The invention is not limited to any particular number of contacting segments 94. For example, the respective values of an electrical parameter may be compared for two or more contacting segments 94. In one aspect, electrical parameter values for one or more pairs of contacting segments 94 may be compared via comparator 102, wherein such pairs of contacting segments 94 that undergo comparison may or may not be arranged opposite, e.g., diametrically opposite, each other.

As a simple, non-limiting example, in FIG. 5A contact sensors 98a and 98b may sense a first value and a second value of an electrical parameter for first and second contacting segments 94a and 94b, respectively; and comparator 102 may be configured for comparing the first value of the electrical parameter for first contacting segment 94a with the second value of the electrical parameter for second contacting segment 94b.

Comparison of the first and second values of the electrical parameter for first and second contacting segments 94a and 94b may indicate contact or lack of contact between first contacting segment 94a or second contacting segment 94b and the patient's body. For instance, a marked discrepancy or mismatch between the electrical parameter values for first and second contacting segments 94a and 94b may indicate lack of contact between one of first and second contacting segments 94a, 94b and the patient's body.

In an embodiment, contact monitoring unit 90 may be configured for simultaneously sensing electrical parameter values for the full complement of contacting segments 94, and comparator 102 may be configured for comparing electrical parameter values for the full complement of contacting segments 94 or for any sub-set of contacting segments 94.

In another embodiment, each contacting segment 94 of system 110 may be configured for contacting the patient's body independently of electrode 120. Each of contacting segments 94 may be capacitively coupled to electrode 120, and system 110 may be configured for sensing a change in voltage of contacting segments 94 responsive to loss of contact of electrode 120 with the patient's body.

In yet another embodiment, system 110 may be configured for sensing at least one electrical parameter or an electrical energy level of electrode 120 during a procedure. For example, electrode 120 may be electrically or logically coupled to one or more components of contact monitoring unit 90, e.g., contact sensing module 96, for sensing current, voltage or other parameter of electrode 120. System 110 may be further configured for comparing the at least one electrical parameter or electrical energy level of electrode 120 with at least one electrical parameter or electrical energy level of one or more of contacting segments 94. Such comparison of the sensed value for the at least one contacting segment 94 with the sensed value for electrode 120 may be indicative of contact or lack of contact between the at least one contacting segment 94 and the patient's body. As may be readily apparent to the skilled artisan, in some embodiments, electrode 120 may lack direct electrical coupling or direct communication with contact monitoring unit 90. As may also be apparent to the skilled artisan, in some embodiments, two or more of the mechanisms described herein for monitoring contact of contacting segments 94 with the patient's body may be combined in a single apparatus or system under the invention.

Although FIG. 5A shows three contact sensors 98a-c, the invention is not to be limited to any particular number of contact sensors, nor to any particular number of contacting segments 94. For the sake of clarity, only those contacting segments 94 that are shown as being electrically coupled to sensing module 96 are specifically denoted by an alpha character (i.e., as 94a-c). In practice, each of contacting segments 94 may be electrically coupled to sensing module 96. Further, while contacting segments 94 are shown in FIG. 5A as being substantially arcuate, system 110 is not to be limited to contacting segments 94 of any particular configuration. For example, in some embodiments one or more of contacting segments 94 may be linear, elongate, oval, square, round, and the like.

In an embodiment, system 110 of FIG. 5A may include from two (2) to twenty (20) or more contacting segments 94, typically from about two (2) to twelve (12) contacting segments 94, often from about four (4) to twelve (12) contacting segments 94, and usually from about four (4) to ten (10) contacting segments 94. Regardless of the configuration of contact monitoring unit 90, an electrical parameter value may be sensed for each of contacting segments 94. Additionally, although electrode 120 is shown in FIG. 5A as being substantially circular, system 110 is not limited to any particular electrode configuration.

Contact monitoring unit 90 may be configured for monitoring contact between each of contacting segments 94 and the patient's body during an electrosurgical procedure. Since contacting segments 94 may be disposed adjacent to electrode 120, contact monitoring unit 90 may also be used to indicate a possible or potential lack of contact between electrode 120 and the patient's body. For example, lack of contact between contacting segments 94 and the patient's body may also indicate a condition that may result in lack of contact between electrode 120 and the patient's body, e.g., an improperly placed dispersive pad. In the event that lack of contact between one or more of contacting segments 94 and the patient's body is indicated by contact monitoring unit 90, power from power supply 20 may be adjusted or shut off, and/or a signal may be provided to alert medical personnel. The medical personnel may then intervene to prevent loss of contact between electrode 120 and the patient.

FIG. 5B schematically represents an electrosurgical system, according to another embodiment of the invention. System 110 of FIG. 5B may include at least one electrode 120, a contacting element 92, and a contact sensing module 96. Contacting element 92 may include a plurality of contacting segments, which are shown as a first opposing pair 94c, 94c′ and a second opposing pair 94d, 94d′; however, other numbers and configurations may be used, substantially as described with reference to FIG. 5A. Each of contacting segments 94c, 94c′, 94d, 94d′ may be configured for contacting a patient's body. Contacting segments 94c, 94c′, 94d, 94d′ may contact the patient's body independently of electrode 120.

Lack of contact between one or more of contacting segments 94c, 94c′, 94d, 94d′ and the patient's body may be detected via contact sensing module 96. Contact sensing module 96 may include a sensor unit 104 and a comparator 102. Comparator 102 may include an analog to digital converter (ADC) 106 in communication with sensor unit 104. Comparator 102 may further comprise a CPU, microprocessor, or microcontroller (not shown), and the like, as is well known in the art. Sensor unit 104 may include one or more sensors (see, for example, FIG. 5A) configured for sensing at least one electrical parameter of each contacting segment 94c, 94c′, 94d, 94d′. Sensor unit 104 may further include a multiplexer, and analog circuitry including protection circuitry for protecting ADC 106 from static discharge or high voltages that might occur around electrode 120 during treatment. An electrical parameter value (such as a value for current, voltage, power, or impedance) of one or more contacting segments 94c, 94c′, 94d, 94d′ may be sensed by sensor unit 104, the sensed value converted by ADC 106 to a digital signal, and comparator 102 may process the digital signal to determine whether one or more of contacting segments 94c, 94c′, 94d, 94d′ lacks contact with the patient's body.

In an embodiment, a parameter such as voltage may be compared between pairs, which may include one or more oppositely disposed pairs, of contacting segments 94c, 94c′ or 94d, 94d′ as an indication of contact, or lack of contact, with the patient's body. Contact sensing module 96 may be in communication with power supply 20 and/or with a signal unit 99 (see, for example, FIG. 3), for cutting power or emitting a warning signal in response to any monitored lack of contact between one or more of contacting segments 94c, 94c′, 94d, 94d′ and the patient's body. In an embodiment, contact sensing module 96 may be integral with power supply 20 (see, for example, FIG. 4).

In an embodiment, contacting element 92 of FIG. 5B may include from two (2) to twenty (20) or more contacting segments 94, typically from about two (2) to twelve (12) contacting segments 94, often from about four (4) to twelve (12) contacting segments 94, and usually from about four (4) to ten (10) contacting segments 94. Contacting segments 94 may be disposed adjacent to electrode 120, essentially as described above with reference to FIG. 5A. Each of contacting segments 94c, 94c′, 94d, 94d′ may be physically isolated or separate from electrode 120, and each of contacting segments 94c, 94c′, 94d, 94d′ may be physically separate from electrode 120, essentially as described above with reference to FIG. 5A. Any electrical energy received from or delivered to the patient's body by contacting segments 94c, 94c′, 94d, 94d′/contacting element 92 will typically be much less than that received or delivered by electrode 120.

Although contacting segments 94c, 94c′ 94d, 94d′ are described with reference to FIG. 5B as being in pairs, groups (other than pairs) of contacting segments 94 may be monitored together, or each contacting segment may be individually monitored, by contact sensing module 96, for contact with the patient's body. Regardless of the configuration of contact monitoring unit 90, an electrical parameter may be sensed for each of contacting segments 94 to indicate or monitor any lack of contact between one or more of the contacting segments and the patient's body.

FIG. 6A schematically represents a dispersive return pad adapted for monitoring contact with a patient, according to another embodiment of the invention. Dispersive return pad 50 may include a plurality of contacting segments 94a-n and at least one return electrode 120a. The at least one return electrode 120a may comprise one or more return electrodes. Return electrode 120a of FIG. 6A may be at least substantially planar and flexible or conformable, and the like. However, many other electrode configurations are also within the scope of the invention. Also, although FIG. 6A shows only a single return electrode 120a, alternative embodiments of the invention may include a plurality of return electrodes 120a of various configurations.

With further reference to FIG. 6A, dispersive return pad 50 may further include a support layer 52. Contacting segments 94a-n and return electrode 120a may be disposed on support layer 52. Support layer 52 may be substantially planar. Each of contacting segments 94 may be physically isolated or separate from electrode 120a, and contacting segments 94 may be physically isolated or separate from each other. Contacting segments 94a-n may be disposed adjacent to return electrode 120a. In this context, “adjacent to” may be used to describe one or more configurations substantially as described with reference to FIG. 5A. As shown, contacting segments 94 may be disposed external to the perimeter, Pₑ, of electrode 120.

In an embodiment, contacting segments 94 may at least partially encircle or surround return electrode 120a. Contacting segments 94a-n may be disposed at or near an edge, or at the periphery, of dispersive return pad 50. As shown, contacting segments 94a-n may be disposed at or near the corners of dispersive return pad 50/support layer 52. Typically, a total amount of any electrical energy received from or delivered to the patient's body by contacting segments 94a-n will be much less than that received or delivered by return electrode 120a. In this regard, it should be clearly understood that contacting segments 94a-n are not intended to function as return electrodes and that for practical purposes contacting segments 94a-n do not function as such.

Dispersive return pad 50, including return electrode 120a and contacting segments 94a-n, may be configured for contacting a patient's body during an electrosurgical procedure. Since contacting segments 94a-n may be disposed adjacent to return electrode 120a to be monitored, lack of contact between contacting segments 94a-n and the patient's body may indicate a condition of dispersive pad 50 that has the potential to result in loss of contact between return electrode 120a and the patient's body. Thus, by the intervention of medical personnel responsive to an indication of lack of contact between contacting segments 94a-n and the patient's body, loss of contact between return electrode 120a and the patient's body may be prevented. Any lack of contact between contacting segments 94a-n and the patient's body may be monitored by one or more of the mechanisms described herein, e.g., with reference to FIGS. 5A-B (supra).

With reference once again to FIG. 6A, in an embodiment contacting segments 94a and 94c may be monitored as one pair, wherein contacting segments 94a and 94c may be at least substantially opposite each other; similarly, contacting segments 94b and 94n may be monitored as another pair. Alternatively, contacting segments 94a and 94b may be monitored as an adjacent, non-opposite pair. Although FIG. 6A shows four contacting segments 94a-n, it is to be understood that other numbers and arrangements of contacting segments 94a-n are also within the scope of the invention. For example, in various embodiments, dispersive return pad 50 may include from about two (2) to twenty (20) or more contacting segments 94a-n, typically from about two (2) to twelve (12) contacting segments 94a-n, often from about four (4) to twelve (12) contacting segments 94a-n, and usually from about four (4) to ten (10) contacting segments 94a-n.

Although contacting segments 94a-n are shown in FIG. 6A as being substantially square, dispersive return pad 50 is not to be limited to contacting segments 94a-n of any particular configuration. For example, in some embodiments one or more of contacting segments 94a-n may be linear, elongate, oval, arcuate, round, and the like. Furthermore, various other configurations or shapes for both return electrode 120a and dispersive return pad 50 are also within the scope of the invention, and the embodiment of FIG. 6A is not to be limited to any particular electrode configuration or return pad geometry.

FIG. 6B schematically represents a treatment face of an electrosurgical instrument, according to another embodiment of the invention. Electrosurgical instrument 30 may include an active electrode 120b. Active electrode 120b may be disposed on or at treatment face 36. Treatment face 36 may be configured for contacting a patient's body during treatment of the patient with electrosurgical instrument 30, and for applying electrical energy to tissue(s) of the patient's body via active electrode 120b. In an embodiment, electrosurgical instrument 30 may comprise a handpiece (not shown), and treatment face 36 may be a component of the handpiece.

Electrosurgical instrument 30 may be used for performing various procedures, including dermatological or various aesthetic procedures. Active electrode 120b is schematically represented as substantially round or circular; however, the embodiment of FIG. 6B may include many diverse active electrode configurations under the invention.

With further reference to FIG. 6B, treatment face 36 may further include a contacting element 92, which may comprise a plurality of contacting segments 94. In an embodiment, contacting segments 94 may be disposed external to the perimeter, Pₑ, of electrode 120b. In an embodiment, contacting segments 94 may at least partially encircle or surround active electrode 120b. Contacting segments 94 may be configured or arranged as diametrically opposed pairs. However, other configurations for contacting element 92/contacting segments 94 are also possible under the invention, e.g., as described herein with reference to other embodiments, or as may be apparent to the skilled artisan in view of Applicant's teaching herein.

Each of contacting segments 94 may be physically isolated or separate from active electrode 120b and from each other. Contacting segments 94 may be disposed adjacent to active electrode 120b for monitoring contact between treatment face 36 and a patient's body. In this regard, “adjacent to” may be used to define the juxtaposition of contacting segments 94 with respect to active electrode 120b substantially as described herein with reference to, for example, FIG. 6A.

Typically, a total amount of any electrical energy received from or delivered to the patient's body by contacting segments 94 will be much less than that received or delivered by active electrode 120b. For example, a first electrical power level of contacting segments 94 may typically be less than a second electrical power level of active electrode 120b. As a non-limiting example, first electrical power level of contacting segments 94 may be less than 50 percent (<50%) of second electrical power level of active electrode 120b, usually less than 10 percent (<10%) of second electrical power level of active electrode 120b, and often less than one percent (<1%) of second electrical power level of active electrode 120b. In this regard, it should be clearly understood that contacting segments 94 are not intended to function as power delivery electrodes.

Contacting segments 94 may be configured for monitoring contact of at least a portion of treatment face 36 with a patient's body or tissue(s) during a procedure. For example, contact of contacting segments 94 with the patient's body during a procedure may be monitored by one or more of the mechanisms described herein with reference to FIGS. 5A-B (supra). None of the embodiments described herein is limited to any particular electrode shape or configuration.

FIG. 7A schematically represents a dispersive return pad for an electrosurgical system, according to another embodiment of the invention. Dispersive return pad 50 may include a support layer 52, a return electrode 60, and a plurality of contacting segments. Only two contacting segments are shown in FIG. 7A, namely a first contacting segment 94a and a second contacting segment 94b, it being understood that dispersive return pad 50 may include up to 20 or more contacting segments 94. Dispersive return pad 50, including return electrode 60 and contacting segments 94a, 94b may be configured for contacting the patient's body, PB. Return electrode 60 may be coupled to power supply 20 (see, e.g., FIGS. 1, 2, and 4) via cable 25b. Dispersive return pad 50 may have other elements, features, and characteristics as described herein for various embodiments of the invention, for example, as described with reference to FIGS. 5A-B and 6A. Any lack of contact between first or second contacting segments 94a, 94b and the patient's body may be monitored by contact monitoring mechanisms as described hereinabove, e.g., with reference to FIGS. 5A-B and 6A.

FIG. 7B schematically represents an electrosurgical instrument for an electrosurgical system, according to another embodiment of the invention. Instrument 30 may include an active electrode 40, a treatment face 36, and a plurality of contacting segments 94a, 94b. Although only two contacting segments are shown in FIG. 7B, electrosurgical instrument 30 may include up to 20 or more contacting segments 94, as described hereinabove. Active electrode 40 may be coupled to power supply 20 (see, e.g., FIGS. 1, 2, and 4) via cable 25a. Active electrode 40 and contacting segments 94 may be disposed on or at treatment face 36; and treatment face 36 may be configured for contacting the patient's body during a procedure, at which time electrical energy may be applied to a target tissue of the patient via active electrode 40. Instrument 30 may have other elements, features, and characteristics as described herein for various embodiments of the invention, for example, as described with reference to FIGS. 5A-B and 6B. Any lack of contact between first or second contacting segments 94a, 94b and the patient's body may be monitored by contact monitoring mechanisms as described hereinabove, e.g., with reference to FIGS. 5A-B, 6B, and 7A.

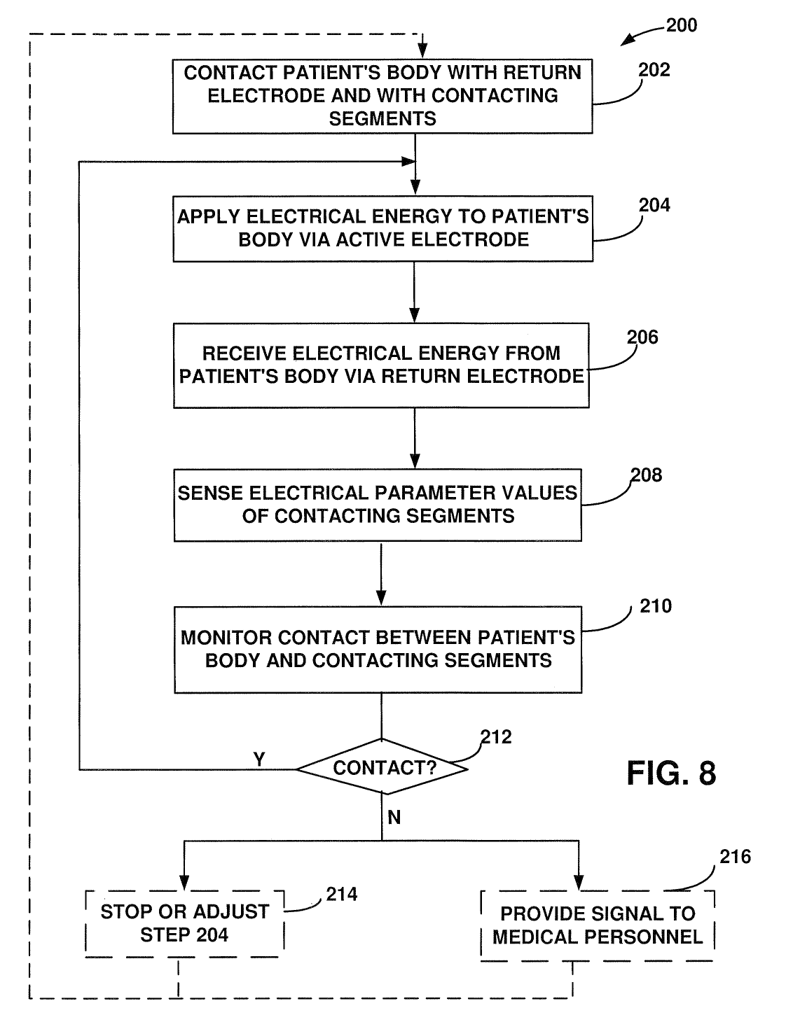

FIG. 8 is a flow chart schematically representing a series of steps involved in a method 200 for monitoring contact between a dispersive return pad and a patient's body during an electrosurgical procedure, according to another embodiment of the invention. Step 202 may involve contacting the patient's body with a plurality of contacting segments of a contact monitoring unit configured for monitoring contact between the contacting segments and the patient's body (see, e.g., FIGS. 5A-6A).

Step 202 may further involve contacting the patient's body with at least one return electrode. The contacting segments may be disposed on a support layer of a dispersive pad at locations adjacent to the return electrode, wherein the support layer may support the return electrode, and step 202 may include contacting the patient's body with the dispersive pad. The contacting segments may be configured such that the contacting segments would contact the patient's body when the dispersive pad is properly placed on the patient's body prior to steps 204 and 206 (infra). In an embodiment, the contacting segments may at least partially encircle or surround the return electrode. Each of the contacting segments 94 may be physically isolated or separate from the return electrode and from each other.

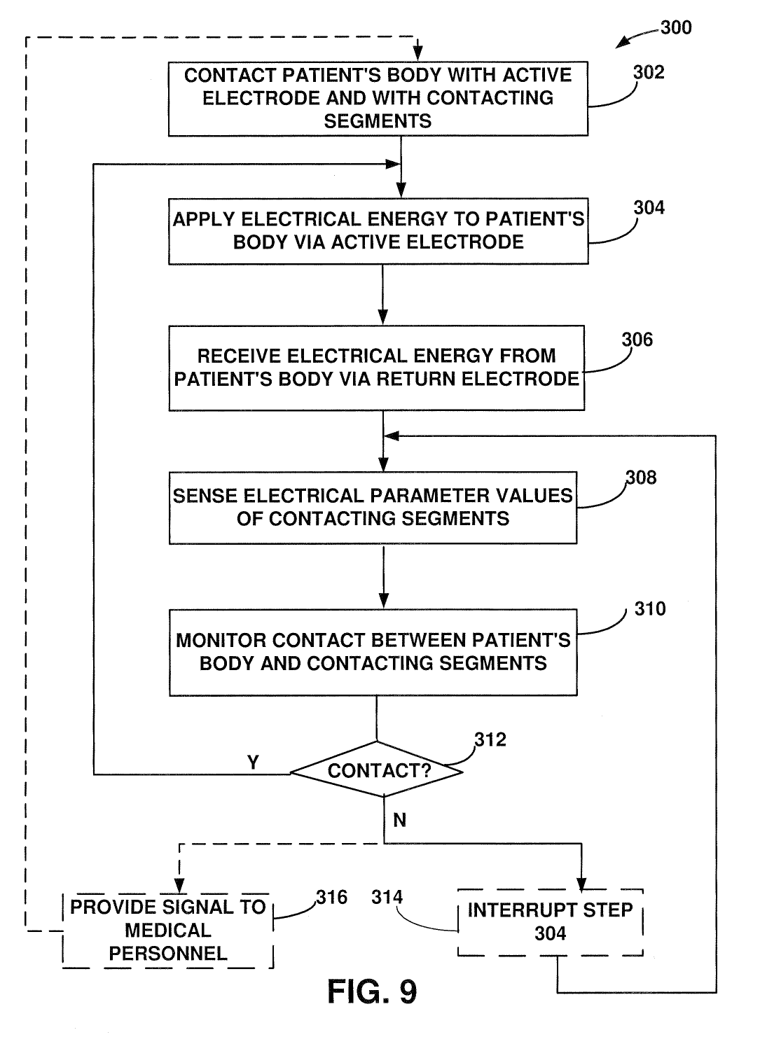

Step 204 may involve applying electrical energy to the patient's body, via an active electrode of an electrosurgical instrument. The active electrode may be disposed on a treatment face of an electrosurgical instrument. The electrosurgical instrument may, or may not, include a plurality of contacting segments disposed adjacent to the active electrode for monitoring contact of the electrosurgical instrument with the patient's body (see, e.g., FIG. 9). For example, in some embodiments both the dispersive return pad and the electrosurgical instrument may be equipped with contacting segments for monitoring their respective contact with the patient's body (see, e.g., FIGS. 7A-B).

Step 206 may involve receiving the electrical energy from the patient's body via the return electrode. Step 208 may involve sensing at least one electrical parameter value of each contacting segment. Step 208 may be performed via one or more contact sensors, wherein each contact sensor may be in communication with one or more of the contacting segments (for example, as described hereinabove with reference to FIG. 5A).

Step 210 may involve monitoring contact between at least one of the contacting segments and the patient's body during a procedure. Contact of the contacting segments with the patient's body may be monitored by one or more of the mechanisms described hereinabove, e.g., with reference to FIGS. 5A-B. For example, step 210 may include comparing a sensed value of an electrical parameter for at least one of the contacting segments with a pre-set or reference value of the electrical parameter, wherein such comparison of the sensed electrical parameter value of the contacting segment(s) with the reference value may indicate contact or lack of contact between the contacting segment(s) and the patient's body.

In another embodiment, step 210 may involve comparing respective sensed electrical parameter values of at least two of the contacting segments, wherein a comparison of a first value of the electrical parameter of a first contacting segment with a second value of the electrical parameter of a second contacting segment is indicative of contact or lack of contact between the contacting segments and the patient's body, e.g., as described with reference to FIGS. 5A-B. Since the contacting segments may be disposed adjacent to the return electrode (see, e.g., FIGS. 5A-B and 6A), lack of contact between the contacting segments and the patient's body may provide advance warning of a situation, e.g., an improperly placed dispersive pad, that could potentially result in loss of contact between the return electrode and the patient's body. In an embodiment, step 210 may involve monitoring contact between the contacting segment(s) and the patient's body as a means for indirectly monitoring contact between one or more portions of the dispersive pad and the patient's body. Contact between the contacting segments and the patient's body according to step 210 may be continuously monitored during steps 204 and 206.

At decision block or step 212, if there is contact between each contacting segment and the patient's body (Y), flow may proceed back to block/step 204 for reiteration. Conversely, if one or more of the contacting segments lacks contact with the patient's body (N), the electrosurgical procedure may be interrupted, and/or flow may proceed to step 214 and/or step 216.

Step 214 of method 200 may involve stopping or adjusting application of electrical energy to the patient's body, e.g., via shutting off, or reducing power output from, the power supply. Alternatively or in addition to step 214, step 216 may involve providing or emitting a signal to alert medical personnel of a possible lack of contact between the electrode and the patient's body. Such a signal may be provided or emitted by a signal unit (see, for example, FIG. 3). Thereafter, if the electrosurgical procedure is incomplete, flow may proceed back to block or step 202, whereby the patient's body may once again be contacted with the contacting segments.

After any corrective action that may be required to properly place, or replace, the dispersive pad on the patient's body, e.g., via the intervention of medical personnel, flow may once again proceed through steps 204-210 for reiteration. By continuously monitoring contact between the contacting segments and the patient's body during steps 204 and 206, loss of contact between the return electrode and the patient's body may be avoided.

In an embodiment, a method for monitoring patient contact with a dispersive return pad may be performed at different stringency levels. For example, at a high level of stringency, the method may be performed substantially as described hereinabove for method 200, decision block or step 212. At a lower level of stringency, contact between the contacting segments and the patient's body may be imperfect, but the procedure may be deemed safe to continue depending on the degree of patient contact with the contacting segments (e.g., a particular number and/or sequence of contacting segments may be in contact with the patient's body). In an embodiment, a level of stringency for performing contact monitoring may be pre-programmed into the electrosurgical system, e.g., according to the nature of the procedure being performed, the configuration of the electrode(s), or other factors.

FIG. 9 is a flow chart schematically representing a series of steps involved in a method 300 for monitoring contact between an electrosurgical instrument and a patient's body during an electrosurgical procedure, according to another embodiment of the invention. Step 302 may involve contacting the patient's body with a plurality of contacting segments of a contact monitoring unit configured for monitoring contact between the contacting segments and the patient's body (see, e.g., FIGS. 5A-B, 6B, and 7B).

Step 302 may further involve contacting the patient's body with at least one active electrode. The contacting segments and the active electrode may be disposed on or at a treatment face of an electrosurgical instrument, and step 302 may include contacting the patient's body with the treatment face. The contacting segments may be configured such that the contacting segments would ordinarily contact the patient's body when the treatment face is correctly placed on the patient's body during step 304 (infra). The contacting segments may be disposed adjacent to the active electrode. In an embodiment, the contacting segments may at least partially surround or encircle the active electrode. Each of the contacting segments may be physically isolated or separate from the active electrode.

Step 304 may involve applying electrical energy to the patient's body. The electrical energy may be applied to the patient's body via the active electrode disposed on or at the treatment face of the electrosurgical instrument, wherein the treatment face may be configured for contacting the patient's body to apply the electrical energy thereto via the active electrode.

Step 306 may involve receiving the electrical energy from the patient's body via a return electrode. The return electrode may be disposed on a support layer of a dispersive pad. The dispersive pad may, or may not, include a plurality of contacting segments disposed adjacent to the return electrode for monitoring contact of the dispersive pad/support layer with the patient's body (see, e.g., FIG. 8). For example, in some embodiments both the electrosurgical instrument and the dispersive return pad may be equipped with contacting segments for monitoring their respective contact with the patient's body.

Step 308 may involve sensing at least one electrical parameter value of each contacting segment. Step 308 may be performed substantially as described hereinabove with reference to FIGS. 5A-B and 8. Step 310 may involve monitoring contact between the contacting segments and the patient's body during the procedure. Contact of the contacting segments with the patient's body may be monitored, e.g., by one or more of the mechanisms described with reference to FIGS. 5A-B, substantially as described for step 210 of method 200 (FIG. 8, supra). Contact between the contacting segments and the patient's body according to step 310 may be continuously monitored during step 304.

At decision block or step 312, if there is contact between each contacting segment and the patient's body (Y), flow may proceed back to block/step 304 for reiteration. Conversely, if one or more of the contacting segments lacks contact with the patient's body (N), flow may proceed to step 314 and/or step 316.

Step 314 may involve interrupting step 304 due to lack of contact between one or more of contacting segments and the patient. Flow may then proceed through steps 308-310 for reiteration. After contact of contacting segments with the patient has been re-established (Y, decision block 312), flow may once again proceed to step 304, whereby application of electrical energy to the patient may be resumed.

In an embodiment, step 314 may result in only occasional or intermittent decreases or cuts in power to the active electrode for short or very short periods of time (e.g., in the millisecond range), wherein such reduction or interruption in power may be imperceptible to both the patient and to medical personnel including an operator of the electrosurgical instrument. Apparatus and procedures for transient interruption of step 304 are known in the art.

Furthermore, a method for monitoring patient contact with a treatment face of an electrosurgical instrument may be performed at different stringency levels. For example, at one level of stringency, the procedure may be continued despite imperfect contact between the contacting segments and the patient's body.

Optional step 316 may involve providing a signal to alert medical personnel of any lack of contact between one or more contacting segments and the patient's body, which is indicative of lack of contact between at least a portion of the treatment face of the instrument and the patient's body. With or without the provision of a warning signal (of step 316), the operator of the instrument may re-establish contact between the contacting segments and the patient's body according to step 302, e.g., as a result of repetitive movement of the treatment face relative to a treatment area of the patient's body, and flow may once again proceed through steps 304-310 for reiteration.

By continuously monitoring contact between the contacting segments and the patient's body, uneven treatment of the target tissue during step 304 may be avoided. Furthermore, such continuous monitoring may also prevent patient burns during step 304.

Methods and apparatus of the invention may find applications in many procedures involving biomedical electrodes other than those specifically described herein. As a non-limiting example, methods and apparatus for electrode contact monitoring as described herein may be used during a broad range of electrosurgical procedures in conjunction with both active and return electrodes of various configurations, shapes, or geometries.

It should be understood that the foregoing relates to exemplary embodiments of the invention, none of the examples presented herein are to be construed as limiting the present invention in any way, and that modifications may be made without departing from the spirit and scope of the invention as set forth in the following claims.

What is claimed is:

1. A system for monitoring contact with a patient's body, comprising:
an electrode configured for contacting the patient's body; and
a contact monitoring unit including a plurality of contacting segments, wherein:
each of said contacting segments is physically separate from said electrode, and
said contact monitoring unit is configured for monitoring contact between each said contacting segment and the patient's body.

2. The system of claim 1, wherein:
each of said plurality of contacting segments is disposed adjacent to said electrode, and
each of said plurality of contacting segments is disposed at a location external to a perimeter of said electrode.

3. The system of claim 1, wherein:
said electrode comprises an active electrode configured for applying electrical energy to a target tissue of the patient's body, and
said contacting segments are disposed adjacent to said active electrode.

4. The system of claim 1, wherein said contact monitoring unit is configured for sensing a value of an electrical parameter for each of said plurality of contacting segments.

5. The system of claim 1, wherein:
said plurality of contacting segments are physically separate from each other, and
each said contacting segment is configured for contacting the patient's body independently of said electrode.

6. The system of claim 1, wherein:
said electrode comprises a return electrode,
said return electrode is disposed on a support layer of a dispersive return pad,
said plurality of contacting segments are disposed on said support layer, and said contacting segments are disposed adjacent to said return electrode.

7. The system of claim 4, wherein:
said electrical parameter comprises at least one of current and voltage, and
lack of contact between any one said contacting segment and the patient's body causes a change in said current or said voltage at said any one contacting segment.

8. The system of claim 4, wherein:
said contact monitoring unit is configured for comparing respective values of said electrical parameter for at least two of said plurality of contacting segments, and
wherein a comparison of said respective values of said electrical parameter is indicative of contact or lack of contact between at least one of said contacting segments and the patient's body.

9. The system of claim 4, wherein:
said plurality of contacting segments includes a first contacting segment and a second contacting segment, and
a mismatch between a first sensed value of said electrical parameter of said first contacting segment and a second value of said electrical parameter of said second contacting segment indicates lack of contact of one of said first and second contacting segments with the patient's body.

10. The system of claim 4, wherein:
said contact monitoring unit is configured for comparing said sensed electrical parameter value for each said contacting segment with a reference value of said electrical parameter, and
wherein comparison of said sensed value for each said contacting segment with said reference value is indicative of contact or lack of contact between each said contacting segment and the patient's body.

11. The system of claim 4, wherein:
said system is configured for sensing a value of said electrical parameter for said electrode, and
said contact monitoring unit is configured for comparing said sensed value for said contacting segments with said sensed value for said electrode, wherein comparison of said sensed value for said contacting segments with said sensed value for said electrode is indicative of contact or lack of contact between said contacting segments and the patient's body.

12. A system for monitoring contact with a patient's body, comprising:
a contact monitoring unit including a plurality of contacting segments; and
an electrode configured for contacting the patient's body, wherein:
each of said contacting segments is disposed adjacent to said electrode,
each said contacting segment is configured for contacting the patient's body independently of said electrode,
said contacting segments are capacitively coupled to said electrode, and
said system is configured for sensing a change in voltage of said contacting segments responsive to loss of contact of said electrode with the patient's body.

13. A dispersive return pad, comprising:
a return electrode configured for contacting a patient's body; and
a contacting element including a plurality of contacting segments, wherein:
said contacting segments are disposed adjacent to said return electrode,
each of said contacting segments is configured for contacting the patient's body,
each of said contacting segments is physically separate from said return electrode, and
said contacting element is configured for monitoring contact between said contacting segments and the patient's body.

14. The dispersive return pad of claim 13, wherein said contacting element comprises from about four (4) to twelve (12) of said contacting segments.

15. The dispersive return pad of claim 13, further comprising:
a support layer, wherein:
said return electrode is disposed on said support layer, and
said contacting segments are disposed on said support layer at a location external to a perimeter of said return electrode.

16. Apparatus for treating a patient, comprising:
an active electrode configured for applying electrical energy to the patient's body; and
a plurality of contacting segments disposed adjacent to said active electrode, wherein:
each of said contacting segments is physically separate from said active electrode, and
said apparatus is configured for monitoring contact between each said contacting segment and the patient's body.

17. The apparatus of claim 16, further comprising:
at least one contact sensor in communication with said plurality of contacting segments, said at least one contact sensor configured for sensing a value of at least one electrical parameter for each said contacting segment; and
a comparator in communication with said at least one contact sensor, said comparator configured for comparing said sensed electrical parameter values,
wherein a comparison of said electrical parameter values is indicative of contact or lack of contact between said contacting segments and the patient's body.

18. The apparatus of claim 16, wherein a first electrical power level of said contacting segments is less than 50 percent (<50%) of a second electrical power level of said active electrode.

19. The apparatus of claim 16, further comprising:
a treatment face configured for contacting the patient's body, wherein:
said contacting segments are disposed on or at said treatment face, and
said contacting segments at least partially surround said active electrode.

20. A method for monitoring an electrosurgical procedure, comprising:
a) contacting the patient's body with an electrode;
b) contacting the patient's body with a plurality of contacting segments, wherein each said contacting segment is physically separate from said electrode; and
c) monitoring contact between each of said plurality of contacting segments and the patient's body.

21. The method of claim 20, wherein:
step c) comprises comparing a sensed value of an electrical parameter of each said contacting segment with a reference value of said electrical parameter, and
wherein said comparing of said sensed electrical parameter value of each said contacting segment with said reference value is indicative of contact or lack of contact between each said contacting segment and the patient's body.

22. The method of claim 20, wherein:
step c) comprises comparing respective values of an electrical parameter for at least two of said plurality of contacting segments, and
wherein said comparing of said respective values of said electrical parameter is indicative of contact or lack of contact between said contacting segments and the patient's body.

23. The method of claim 20, wherein said step c) comprises comparing a sensed voltage for at least two of said plurality of contacting segments.

24. The method of claim 20, wherein:
said electrode comprises an active electrode, and
the method further comprises: d) applying electrical energy to the patient's body via said active electrode, wherein:
said contacting segments are disposed adjacent to said active electrode, and
a first electrical power level of said contacting segments is less than 50 percent (<50%) of a second electrical power level of said active electrode.

25. The method of claim 20, wherein:
said electrode comprises a return electrode,
step a) comprises contacting the patient's body with a dispersive return pad, wherein said dispersive return pad includes said return electrode, and
said dispersive return pad further includes a support layer, wherein:
said return electrode is disposed on said support layer,
said plurality of contacting segments are disposed on said support layer,
said plurality of contacting segments are physically separate from each other,
said plurality of contacting segments are disposed adjacent to said return electrode, and
said plurality of contacting segments at least partially surround said return electrode.
